(19) United States
(12) Patent Application Publication
(10) Pub. No.: US 2014/0356460 A1
(43) Pub. Date: Dec. 4, 2014

(54) Use of hypertonic saline to draw fluid out of swollen tissue and relieve nasal congestion

(71) Applicant: Matthew Lutin, Carlsbad, CA (US)

(72) Inventor: Matthew Lutin, Carlsbad, CA (US)

(21) Appl. No.: 13/986,706

(22) Filed: May 28, 2013

(51) Int. Cl. A61K 33/14; A61K 9/08

(52) U.S. Cl. CPC A61K 33/14; A61K 9/08
USPC 424/680

(57) ABSTRACT

The optimum concentration of salt in solutions to combat congestion of the mucosa is a relatively narrow range. This use of in hypertonic saline solutions for treatment of congestion of the mucosa having a concentration within the range of 2.3% to and including 2.7% w/v sodium chloride/water with the most preferred concentration being 2.4% to and including 2.6% w/v of sodium chloride in water brings maximum relief of congestion and for cleansing membranes without causing irritation or discomfort.

This application is a continuation-in-part of U.S. patent application Ser. No. 13/385,835, filed Mar. 9, 2012, now pending.

### FIELD AND BACKGROUND OF THE INVENTION

This application relates to relief of discomfort arising from congestion whether due to infection, allergic response or simple mechanical irritation. While the use of hypertonic solutions to relieve nasopharyngeal congestion has been known, the usual concentration on the market contains approximately 3% sodium chloride. Saline solutions containing sodium chloride/water commonly on the market are 0.65%, 0.9%, and 3% sodium chloride. However, it has now been found that the range at which hypertonic saline will provide most effective response without a rebound congestion is quite narrow, being from 2.3% to and including 2.7% w/v sodium chloride/water, with the most preferred concentration being 2.4% to and including 2.6% w/v of sodium chloride in water. The method of the invention does not require use of a nasal cup or other complicated equipment. Applications by mist, drops or as a flowing liquid over the membranes are all effective.

Pain and discomfort are experienced when the mucous membrane swells as it becomes inflamed, blocking the drainage of fluid from the sinuses into the nose and throat. Mucus and fluid build up inside the sinuses, causing pressure and pain. The discomfort and problems related to some prior treatments are further complicated because bacteria are more likely to grow in sinuses that are unable to drain properly. Bacterial infection then further increases inflammation and pain. Similarly, the eyes may also be damaged by exposure to contaminants or infection which cause irritation or permanent damage to the eye.

U.S. Pat. No. 6,645,532 to Lutin teaches use of hypertonic saline for preventing discomfort in the ears using hypertonic solution in a most preferred range of 2.4% to and included 2.6% w/v sodium chloride/water. The method of that patent requires that the individual retain the solution for at least 5 seconds, then expel the saline under pressure to avoid or remedy discomfort arising from obstruction/congestion in the Eustachian tubes. The instant invention for relief of nasal congestion does not require expelling the saline under pressure, since there is no need to open the Eustachian tubes for relief of simple nasal congestion.

U.S. Pat. No. 6,899,903 to Quillin teaches a composition for cleansing the sinuses using a solution containing Baptisia tincturia colloidal silver, grapefruit seed extract and oregano juice and, further, incorporating 0.01% to 5% sodium chloride. The lone example uses a solution containing 0.75% sodium chloride. However, as indicated below, some of these concentrations of sodium chloride in water are ineffective and some cause undue irritation. The lower concentrations of sodium chloride tested as disclosed herein were essentially ineffective for purposes of relieving congestion, and, in fact, the studies showed that the lower concentrations of sodium chloride hydrate the tissue, increasing congestion.

U.S. Pat. No. 6,258,372 to Jones teaches use of a solution containing xylitol/xylose and between 0.45% and 0.95% sodium chloride with a preferred concentration of 0.65% sodium chloride, a concentration which is far less effective for relieving congestion than the preferred concentration disclosed and claimed herein. (The Jones reference teaches preferred concentrations of sodium chloride which are hypotonic,)

U.S. Pat. No. 5,897,872 to Picciano teaches a nasal moisturizing saline solution containing iodine and hypotonic 0.65% sodium chloride.

Rabago, et al., “Efficacy of Daily Hypertonic Saline Nasal Irrigation Among Patients with Sinusitis: A Randomized Controlled Trial”, Journal of Family Practice, December 2002, Vol. 51, No. 12: 1049-1055 teaches use of 2% hypertonic saline as a nasal irrigation for treatment of sinusitis. The method of Rabago, et al. involves use of a nasal cup. That concentration of sodium chloride has been shown to be less useful in the testing as shown below when using the much simpler methods of the invention.

### SUMMARY OF THE INVENTION

It is the purpose of this invention to relieve discomfort arising from congestion of the mucous membranes of the nasal passages or irritation of the eye. While there are many references which teach use of saline to relieve congestion, it has now been found that a very narrow range of salt concentration provides optimal relief without discomfort. Hypertonic saline of 2.3% to and including 2.7% (preferably 2.4% to and including 2.6%) gives optimum results without causing irritation or with minimal irritation. When lower levels of salinity than taught herein are used, the treatment is less effective. When washes with higher salinity are used, there is often irritation to the membranes and the initial effect may be countered with a rebound swelling. The methods and equipment used may vary. However, any means which delivers the saline to the membranes is acceptable.

### DETAILED DESCRIPTION OF THE INVENTION

As indicated in the Background of the Invention, many references teach a wide range of salt concentrations of saline without any suggestion that the concentration used as described herein would have any improved benefit. Moreover, none of the references teach the simple application of saline within the range taught herein as a means of providing relief from congestion of the mucous membranes. Unlike the method taught in U.S. Pat. No. 6,645,532 to Lutin for relief from discomfort in the ears, there is no need for the patient suffering from simple congestion of the mucous membranes to exert pressure by blocking the nose when expelling the saline. In fact, mere washing of the membranes with the saline provides relief and is preferred. The saline may be administered in any way that causes the solution to contact the mucous membrane. For example, the saline may be delivered by drops, sprays, in a mist, by syringe, such as an ear syringe, or by pump. Pressure in administration is not required and pressure in expelling fluid is not necessary.

The use of solutions in the salt concentrations taught herein has proven to be especially useful for application to eyes that have become contaminated and irritated by contaminants such as smoke or dust. Use of a mist or wash at the preferred concentration of salt as taught herein proved especially useful for cleansing the surface of the eye.

The objective in choosing a solution was to find the concentrations at which there was least irritation with good relief of congestion. It was noted that at concentrations above 2.7% there was an immediate relief of congestion; however, when higher concentrations of salt were used there appeared to be a rebound increase in congestion following immediate (short term) relief.

### Materials and Methods

Saline solutions of varying strengths were prepared. The solution was administered as a mist through the nose, and then allowed to flow out of the nose. The following shows the results obtained with various concentration of salt (w/v salt/water).
No irritation −
Some irritation +
Moderate irritation ++
Severe irritation +++
Effectiveness was determined by subjective relief of congestion

### Hypertonic Solutions

2.1%—No irritation − . . . Less effective
2.2%—No irritation − . . . Less effective
2.3%—No irritation − . . . Less effective
2.4%—No irritation − . . . Most effective
2.5%—No irritation − . . . Most effective
2.6%—No irritation − . . . Most effective
2.7%—Some irritation + . . . Most effective
2.8%—Some irritation +
2.9%—Moderate irritation ++
3.0%—Moderate irritation ++
3.1%—Severe irritation +++
3.2%—Severe irritation +++

The concentrations for use on mucous membranes of the nasal tract are also preferred for use in the eye. The application of the solution containing 2.6% sodium chloride as a mist to the eye of a firefighter who had been exposed to contaminants believed to contain asbestos resulted in dislodging of particles, cleansing and relief of irritation.

Many causes lead to congestion and irritation of the exposed surfaces of the respiratory tract and the eye. Colds commonly trigger congestion, but any factor that causes the mucous membrane to become inflamed may lead to sinusitis. Many people with allergies suffer congestion (allergic rhinitis). Congestion arising from allergies often leads to recurring and/or chronic inflammation of the mucous membranes which can give rise to sinus infections. In fact, any condition which blocks the nasal passages increases the risk of sinusitis.

Inflamed, congested mucous membranes can also give rise to snoring. The use of saline of the preferred concentrations in accord with the methods of the invention often decreases snoring. The solution pulls fluid from the mucosa and also flushes out germs, contaminants, and pollutants (pollen/dust/sand/soot/smoke, etc.) from the mucus membranes. Mechanical obstruction such as that caused by deviated septum is also a cause of snoring and of irritation to the mucosa. Any obstruction increases likelihood that irritation of the mucosa will occur.

The use of the solutions in accord with the methods of the invention provides means of prophylaxing against congestion arising from exposure to dust, smoke and other environmental irritants. For example, cleaning the mucous membranes with preferred solutions taught herein after exposure to dust such as that encountered in mining, milling, farming or construction trades can prevent congestion and infection of the mucous membranes. The methods of the invention are particularly useful for cleansing the mucous membranes following exposure to smoke and other contaminants that are routinely encountered by fire fighters and other first responders. Dislodging of fine sand, smoke, ash and soot are important in preventing discomfort and disease.

The solutions may be applied to the mucous membranes by any means which causes the solution to contact the membranes, such as aerosol, dropper, pump, ear syringe or bag on valve (BOV) containers . A relatively new means of application, the nasal mist pump, has increased both sinus tissue area exposure and the amount of liquid solution being dispensed, resulting in a most effective treatment for reducing sinus congestion. In many instances the low pressure nasal mist pump is the best device for administration of the hypertonic solutions. Additionally, the nasal mist pump is a preferred method of administration because it can be loaded with solution, sealed and sold with the solution protected from exposure to contaminants. These pumps provide a means of thoroughly bathing the membranes in the solution. The use of the nasal mist pump was also quite useful for cleansing the eye of contaminants.

When the solution is administered to the nasal mucosa as a stream by syringe, it is best to have the head bent over a basin or sink so that the solution may be allowed to flow out through the nose and mouth. When using a pump, dropper or aerosol (most convenient ways for administration) the solution can simply flow into the nose and out again to cleanse and dehydrate the membranes contacted.

Unlike with the prior invention of Lutin to prevent discomfort in the ears, there is no need to hold the nose while applying pressure. In fact, such application of pressure against a closed nose would not be appropriate in treatment, especially the treatment of children.

The compositions of the invention need not contain a preservative. In fact, preservative free hypertonic saline at 2.3% to and including 2.7% w/v (preferably 2.4% to and including 2.6% w/v) salt in water is quite effective and has not presented any problem in use so long as the solution is free of contamination. It should be noted that in any case where hypersensitivity or outright allergy is a cause of congestion, it is preferred that the solution be as free of additives as possible.

Sea salt sold commercially usually has the same amount sodium chloride as commercially sold salt from other sources. However, there are some times contaminants in sea salt. If such contaminants are present, the amount of sea salt required to deliver the full amount of sodium chloride at the w/v indicated herein will vary. Hence, it should be stipulated that the amount of sea salt used should be sufficient to provide the appropriate concentration of sodium chloride and such amount will usually depend on the amount of contaminants in the sea salt.

1-20. (canceled)

21. A method of relieving irritation or of removing contaminants from the eyes comprising the steps of application of a hypertonic saline solution containing 2.3% to 2.7% by weight of sodium chloride relative to total volume of solution wherein the saline is administered by mist pump.

22. The method of claim 21 wherein the solution contains sodium chloride concentration of 2.4% to 2.6% by weight of sodium chloride relative to total volume of solution.
